(19) United States
(12) Patent Application Publication
(10) Pub. No.: US 2025/0339490 A1
(43) Pub. Date: Nov. 6, 2025

(54) TOPICAL OCULAR DELIVERY OF CYCLOSPORIN

(71) Applicant: LYOTROPIC DELIVERY SYSTEMS LTD., Jerusalem (IL)

(72) Inventors: Nissim GARTI, Ramat HaSharon (IL); Sharon GARTI-LEVI, Modi'in (IL); Rotem EDRI, Eilat (IL); Rawan MUSLEH, Jerusalem (IL)

(73) Assignee: LYOTROPIC DELIVERY SYSTEMS LTD., Jerusalem (IL)

(21) Appl. No.: 18/995,294

(22) PCT Filed: Jul. 6, 2023
(86) PCT No.: PCT/IL2023/050699

§ 371 Date: Jan. 16, 2025

(30) Foreign Application Priority Data
Jul. 20, 2022 (IL) 294896

(51) Int. Cl. A61K 38/13; A61K 9/00; A61K 9/107; A61K 47/10; A61K 47/14; A61K 47/32; A61K 47/34; A61K 47/38; A61K 47/44

(52) U.S. Cl. CPC A61K 38/13; A61K 9/0048; A61K 9/1075; A61K 47/10; A61K 47/14; A61K 47/32; A61K 47/34; A61K 47/38; A61K 47/44

(57) ABSTRACT

The present disclosure concerns formulations for topical ocular delivery of high concentrations of cyclosporin. The formulations comprise at least 0.1 wt % cyclosporin carried in a plurality of nanostructures in the form of nanodroplets dispersed in an aqueous continuous phase.

### TECHNOLOGICAL FIELD

The present disclosure concerns ophthalmic formulations of topical delivery of cyclosporin to the front of the eye.

### BACKGROUND

Cyclosporins are well known for treatment of various ocular conditions. Cyclosporins are cyclic oligopeptides from the family of anti-calcineurins, and have immunosuppressive and anti-inflammatory activity. Ocular drop formulations of cyclosporin A are widely used to treat various ocular conditions, such as vernal keratoconjunctivitis, corneal transplant rejection, dry eye syndrome (which is resistant to first line treatment), and many other indications.

The high hydrophobicity of cyclosporin has made it difficult to stably formulate into ophthalmic formulations which require high concentrations of cyclosporin. Such formulations were reported to be based on high concentrations of oily components, which are, by themselves, irritants and less tolerable to patients. Further, as high concentrations of cyclosporin have, up to date, been problematic to physically stabilize and capture within formulations, high irritancy of highly-loaded cyclosporin of various formulations was reported. Hence, to date, most commercial formulations contain relatively low concentration of cyclosporin, typically up to 0.05 wt %.

### GENERAL DESCRIPTION

The present disclosure provides ophthalmic topical formulations, particularly in the form of eye drops, which contain high concentrations of cyclosporin, with minimal irritation effects and high active delivery capability. Such formulations can be used, for example, for treatment of front of the eye conditions, such as dry eye disorder.

The formulations of this disclosure contain at least 0.1 wt % of cyclosporin, and are formulated as stable nanostructures, homogenously dispersed in an aqueous phase, in which the cyclosporin is captured and stabilized within the nanostructures. As the nanostructures contain very low amounts of oily components, however still enables capturing of cyclosporin therein due to its unique combination of components, minimal irritation to the eye is observed, while permitting delivery of high effective doses of cyclosporin to the eye. The inventors have surprisingly found that utilizing a combination of at least two non-ionic hydrophilic surfactants enables physical stabilization of high loads of the highly lipophilic cyclosporin within the nanostructure while maintaining very low amounts of oil.

Thus, in one of its aspects, the present disclosure provides an ophthalmic formulation that comprises plurality of nanostructures dispersed in an aqueous continuous phase, the nanostructures being in the form of droplets having an average diameter of at most 50 nm, the nanostructures comprise:
a) cyclosporin in a concentration of at least 0.1 wt % of the formulation,
b) at least two non-ionic hydrophilic surfactants,
c) at least one oil in a concentration of at most 2 wt % of the formulation, and
d) at least one co-surfactant.

The formulations of this disclosure are designed for ophthalmic delivery of cyclosporin, i.e. delivery of cyclosporin to one or more part of the eye, for example to the cornea, conjunctiva, aqueous humor, iris, vitreous humor, ciliary body, anterior chamber, posterior chamber, etc. The formulation is preferably a topical formulation in the form of a solution or suspension of said nanostructures in said continuous aqueous phase.

The nanostructures are droplets composed at least of said at least one oil, non-ionic hydrophilic surfactants, and at least one co-surfactant, that capture and stabilize cyclosporin. The nanostructures are typically in the form of vesicles, having an average diameter of at most 50 nm (nanometers), in which the non-ionic hydrophilic surfactants and co-surfactants form an interface between the continuous aqueous phase and the oil core. Without wishing to be bound by theory, the cyclosporin is predominantly located at the interface, where it is physically captured between the heads of the surfactants and co-surfactants interacting via hydrogen bonds and dipole-dipole interactions, thereby stabilizing it within the nanostructures.

The term average size refers to the arithmetic mean of measured diameters of the droplets. Where the droplets are not spherical, the calculation of the average size is based on an equivalent sphere about the largest dimension of the particles.

By some embodiments, the droplets are substantially mono-disperse. The formulations are typically transparent (or substantially transparent) due to their mono-dispersed submicronic nanostructures size, maintaining their transparency for a prolonged period of time. This permits easy detection of changes in the formulation's stability (as phase separation, bioactive precipitation, and/or coalescence of oil droplets will cause detectable clouding).

Cyclosporin is a cyclic oligopeptide from the family of anti-calcineurins. Cyclosporin A is a cyclic hydrophobic undecapeptide that contains 7N-methyl-amino acid residues and the amino acid (4R)-4-([E]-2-butenyl)-4-N-methyl-(L)-threonine (MeBmt), as shown in formula (I):

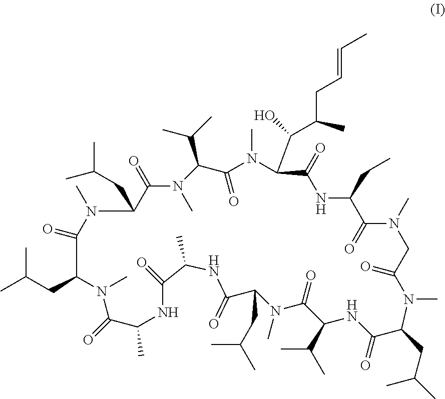 [SMILES: C/C=C/C[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@H](CC(C)C)C(=O)N[C@@H](C)C(=O)N[C@H](C)C(=O)N(C)[C@@H](CC(C)C)C(=O)N(C)[C@H](CC(C)C)C(=O)N(C)[C@@H](C(C)C)C(=O)N1C]

Within the context of the present disclosure, the term cyclosporin refers to cyclosporin A, salts, derivatives and analogues thereof.

In some embodiments, cyclosporin is cyclosporin A.

As noted, the formulations of this disclosure are highly-loaded with cyclosporin. The formulations of this disclosure, due to their unique compositional balance, enable stably loading the formulation with cyclosporin at concentrations well beyond its solubility limit in water (which is 27.67 μg/ml, or ˜0.027 wt % at 25° C.). In some embodiments, the cyclosporin is in a concentration of at least about 0.1 wt %, at least about 0.15 wt %, at least about 0.2 wt %, at least about 0.25 wt % or even at least about 0.3 wt % of the formulation (e.g. about 0.5 wt %).

The inventors have found that a combination of two or more non-ionic hydrophilic surfactants enables the high loading of cyclosporin into the formulation and stabilization thereof for prolonged period of time. In the formulations of this disclosure, the balance of ingredients permits not only high load and capturing of cyclosporin in the formulation, but also obtaining both kinetic and thermodynamic stabilization of the formulation, hence permitting a long shelf life with minimal phase separation, sedimentation and/or undesired discharge of cyclosporin out of the nanostructures.

The term non-ionic hydrophilic surfactant(s) refers to surface-active agents which are not electrically charged, and have a hydrophilic head group and lipophilic tail(s) that are capable of arranging into nanostructures in an aqueous medium. The inventors have found that a combination of two or more such non-ionic hydrophilic surfactants are capable of forming stable nanostructures and solubilize cyclosporin into the nanostructure in relatively high concentrations. By tailoring the composition of the nanostructures, entrapment of cyclosporin between the surfactants tails is obtained, thereby solubilizing it predominantly within the interface, and possibly also within the oil core. The particular combination is based on two non-ionic hydrophilic surfactants which are structurally distinct in the geometry of their head groups and capable of forming head-groups complex. Where one of the surfactants has a linear hydrophilic head, the other has bulky head. Such combination spaces the nanostructures interface, allowing the entrapment of cyclosporin (attributed to the bulky heads) while maintaining the curvature and integrity of the interface (attributed to the linear heads).

According to some embodiments, the at least two non-ionic hydrophilic surfactants comprise at least one first non-ionic hydrophilic surfactant selected from ethoxylated fatty acids, and at least one second non-ionic hydrophilic surfactant selected from ethoxylated castor oil and hydrogenated derivatives thereof.

According to some embodiments, the first non-ionic hydrophilic surfactants can be selected from ethoxylated fatty acids (polyoxyethylene stearates, polyoxyethylene oleates, polyoxyethylene caprylate/caprate, polyoxyethylene laurate etc.), ethoxylated alkyl ethers (polyoxyl cetyl ether, polyoxyethylene lauryl ether, polyoxyl cetostearyl ether, polyoxyl oleyl ether, polyoxyl stearyl ether etc.), ethoxylated monoglycerides, and combinations thereof. By some embodiments, the second non-ionic hydrophilic surfactants can be selected from polyoxyethylene castor oil (polyoxyl 35 castor oil, polyoxyl 40 hydrogenated castor oil, polyoxyl 60 hydrogenated castor oil, polyoxyl 60 castor oil, polyoxyl 100 castor oil, polyoxyl 100 hydrogenated castor oil, polyoxyl 200 castor oil, polyoxyl 200 hydrogenated castor oil, etc.), polyoxyethylene sorbitan fatty acid esters (polysorbate 20, polysorbate 60, polysorbate 80, etc.) and combinations thereof.

According to some embodiments, the weight ratio (w/w) of the first non-ionic hydrophilic surfactants to the second non-ionic hydrophilic surfactants in the formulation ranges between about 1:1 and 1:30. By some other embodiments, the weight ratio (w/w) of the first non-ionic hydrophilic surfactants to the second non-ionic hydrophilic surfactants ranges between about 1:1 and 1:28.

By some embodiments, the total concentration of the non-ionic hydrophilic surfactants in the formulation ranges between about 1 wt % and about 7 wt %.

The formulations comprise at most 2 wt % oil. By some embodiments, the at least one oil is present in the formulation in a concentration of no more than 0.7 wt %. The relatively low content of oil allows the high loading capacity of cyclosporin on the one hand, and on the other hand serves as stabilizer of the nanostructures at high temperatures in the presence of cyclosporin. At high temperatures the polar moieties of cyclosporin are directed towards the core of molecule, and thus reduce the interaction with the hydrophilic heads of the surfactants.

The term oil refers to an agent which is immiscible in water and is capable of forming distinct domains when introduced into an aqueous liquid. In some embodiments, the at least one oil is selected from acylglycerides of fatty acids including triacetin, tributyrin, tricaprylin, triolein, medium chain triglyceride and mixed fatty acids triglycerides, olive oil, sesame oil, soybean oil, canola oil, castor oil, partially or fully hydrogenated castor oil, paraffin oil, mineral oil, non-saponified fatty derivatives, alkyl alcohols including oleyl alcohol, dodecyl alcohol, terpenoids, and combinations thereof.

According to some embodiments, the weight ratio between the total non-ionic hydrophilic surfactants and oil in the formulation ranges between about 5:1 and about 50:1.

As noted, the formulation also comprises at least one co-surfactant. Co-surfactant should be understood to encompass any lipophilic, hydrophilic or amphiphilic agent, different from said non-ionic hydrophilic surfactants, which contributes (together with the surfactants) to lowering of the interfacial tension between the oily phase and the aqueous phase to almost zero (or zero) allowing for the formation of thermodynamically stable nanostructures. Hence, the combination of surfactants and co-surfactants permits stabilization of the nanostructures both kinetically and thermodynamically.

According to some embodiments, the co-surfactant is a hydrophilic co-surfactant or an amphiphilic co-surfactant.

By some embodiments, the at least one co-surfactant is at least one polyol. Polyols are alcohols containing at least 2 hydroxyl groups.

By some embodiments, the at least one co-surfactant is selected from polyethylene glycol 200, polyethylene glycol 400, polyethylene glycol 600, propylene glycol, polypropylene glycol, diethylene glycol monoethyl ether (Transcutol), and combinations thereof.

According to some embodiments, the at least one co-surfactant is present in the formulation in a concentration ranging between about 0.5 wt % and about 5 wt %.

According to other embodiments, the weight ratio between the non-ionic hydrophilic surfactants and the co-surfactants ranges between about 1:1 and 5:1.

It was surprisingly found by the inventors, that the combination of low amount of oil, the at least two non-ionic hydrophilic surfactants and the at least one co-surfactant permits stabilization of cyclosporin at high loads within the formulation at ambient temperatures and cold storage temperatures (e.g. 4° C.), while when warming the formulation to ca. 35-40° C., cyclosporin becomes more hydrophobic and is predominantly stabilized by the surfactants tail and oil. Upon contact with eye epithelium, cyclosporin that was held by the nanostructure is available to be released due to the merging of the physiological membrane and the nanostructure. Hence, formulations of this disclosure were found to be highly stable at storage temperatures, and having increased availability of cyclosporin after administration.

By some embodiments, the nanostructures also comprise at least one solvent. The solvent is an organic solvent, typically polar, that is water miscible and is suitable for assisting the solubilization of cyclosporin into the nanostructure, as well as for adjusting the osmolarity of the system. The introduction of at least one such solvent into the formulation can facilitate full coverage of the interface by the hydrophilic surfactant at high water dilutions of the formulation. In other words, the use of at least one solvent alters the effective critical packing parameter (ECPP) of the interface, facilitating the control of the hydrophilicity/hydrophobicity of the surfactants, depending on the amount of water in the formulation, thus increasing stability of the formulation.

According to some embodiments, said at least one solvent is selected from glycerol, ethanol, methanol, propanol, isopropanol, diethanolamine, triethanolamine, and combinations thereof.

By some embodiments, the formulation comprises said at least one solvent in a concentration ranging between about 0.05 wt % and about 1.5 wt %.

By some other embodiments, the weight ratio between the non-ionic hydrophilic surfactants and the solvents ranges between about 5:1 and 35:1.

In order to increase residence time of the formulation in the eye, the formulation, by some embodiments, further comprises at least one film forming agent in the aqueous phase. The term film forming agent (or film former) refers to a substance that can increase the viscosity of the formulation and temporarily form a thin film over the external mucosal membrane of the eye to delay evacuation of the nanostructures from the eye by the lacrimal fluid. According to some embodiments, said at least one film forming agent is selected from polyvinyl pyrrolidone, block copolymers of polyoxypropylene and polyoxyethylene (poloxamers), carboxymethyl cellulose and salts thereof, hydroxypropylmethylcellulose (HPMC), poly(vinyl alcohol), poly(acrylic acid), hydrocolloids such as xanthan gum, and combinations thereof.

By some embodiments, the concentration of said at least one film forming agent in the formulation is up to about 0.75 wt %.

In some embodiments, the formulations may further comprise various additives approved for ophthalmic uses, such as pH adjusting agents and buffers, neutralizing agents, emollients, humectants, preservatives, antioxidants, etc.

The ophthalmic formulations of this disclosure can be prepared from a concentrated form, typically substantially water free concentrated, that are dilutable by an aqueous medium. This permit forming a concentrate which is stable for prolonged periods of time, which lacks a microorganisms' life-supporting environment, and is readily dilutable for obtaining the nanostructures.

Thus, by another one of its aspects, the present disclosure provides a cyclosporin concentrate formulation suitable for preparing the ophthalmic formulation as described herein, the concentrate comprises:
a) cyclosporin in a concentration of at least 0.5 wt % of the concentrate,
b) at least two non-ionic hydrophilic surfactants,
c) at least one oil in a concentration of at most 12 wt % of the concentrate, and
d) at least one co-surfactant.

By some embodiments, the at least two non-ionic hydrophilic surfactants comprise at least one first non-ionic hydrophilic surfactant selected from ethoxylated fatty acids, and at least one second non-ionic hydrophilic surfactant selected from ethoxylated castor oils and hydrogenated derivatives thereof.

In some embodiments, the weight ratio of the first non-ionic hydrophilic surfactants to the second non-ionic hydrophilic surfactants ranges between about 1:1 and 1:30.

By some embodiments, the at least one oil is present in the concentrate in a concentration of no more than 7 wt %.

By some other embodiments, the total concentration of the non-ionic hydrophilic surfactants in the concentrate ranges between about 20 wt % and about 75 wt %.

According to some embodiments, said at least one co-surfactant is present in the concentrate in a concentration ranging between about 20 wt % and about 45 wt %.

By some embodiments, the concentrate further comprises at least one solvent. In such embodiments, the concentrate comprises said at least one solvent in a concentration ranging between about 1 wt % and about 10 wt %.

The concentrate is essentially devoid of water, i.e. comprises up to about 5 wt % water. By some preferred embodiments, the concentrate is water-free.

A further aspect of this disclosure provides a method of preparing the ophthalmic formulation as described herein, the method comprises mixing the concentrate described herein with an aqueous dispersing medium, thereby obtaining plurality of nanostructures formed from said concentrate and dispersed in an aqueous continuous phase formed from said aqueous dispersing medium.

By some embodiments, said aqueous dispersing medium comprises at least one film forming agent.

By other embodiments, the aqueous dispersing medium comprises at least one buffering agent.

According to some embodiments, said concentrate is mixed with said aqueous dispersing medium in a weight ratio ranging between about 1:5 and 1:25.

By some embodiments, said mixing is carried out under conditions using mechanical rotor or magnetic stirring applying only mild to moderate shear. The system does not need to be subjected to high shears applied by homogenization, intense sonication, fluidizing techniques, etc. Hence, by some embodiments, the mixing is carried out under conditions preventing development of high shear forces in the mixture.

By another aspect, the present disclosure provides a kit for preparing the ophthalmic formulation described herein, the kit comprises at least one first container containing the concentrate described herein, at least one second container containing an aqueous dispersing medium; and instructions for use.

The first and second containers may be independently rigid, semi-rigid or flexible, and may have suitable form. The first and second containers may comprise the concentrate and the aqueous dispensing medium, respectively, in amounts suitable for preparation of a single dose of ophthalmic formulation or for multiple doses thereof.

By some embodiments, the first and second containers are integrally formed and configured for mixing said concentrate and aqueous dispersing medium upon user demand (for example by having the content of one of the containers being introducible into the other container or by having a mixing zone in which the content of the containers can be conveniently mixed).

By another aspect, there is provided an ophthalmic formulation as disclosed herein for use in treating a front of the eye disease or condition.

Another aspect provides a method of treating a front of the eye disease or condition, comprising administering an effective amount of an ophthalmic formulation described herein to a subject in need thereof.

By some embodiments, front of the eye disease or condition is selected from dry eye disease, dry and wet age-related macular degradation, cataract, diabetic retinopathy, glaucoma, amblyopia, and strabismus.

As known, the effective amount for purposes herein may be determined by such considerations as known in the art. The amount must be effective to achieve the desired therapeutic effect, depending, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, the effective amount depends on a variety of factors including a variety of pharmacological parameters such as half-life in the body, on undesired side effects, if any, on factors such as age and gender, and others.

The term treatment or any lingual variation thereof, as used herein, refers to the administering of a therapeutic amount of the formulations of the present disclosure which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease from occurring or a combination of two or more of the above.

As used herein, the term about is meant to encompass deviation of ±10% from the specifically mentioned value of a parameter, such as temperature, concentration, etc.

Unless otherwise specifically indicated, all concentrations disclosed herein are provided as weight percentage, wt %, out of the weight of the ophthalmic formulation or the concentrate formulation, as the case may be.

Whenever a numerical range is indicated herein, it is meant to include any cited numeral (fractional or integral) within the indicated range. The phrases ranging/ranges between a first indicate number and a second indicate number and “ranging/ranges from” a first indicate number “to” a second indicate number are used herein interchangeably and are meant to include the first and second indicated numbers and all the fractional and integral numerals therebetween.

Unless the context requires otherwise, the word comprise, and variations such as “comprises” and “comprising”, will be understood to imply the inclusion of a stated integer or step or group of integers or steps but not the exclusion of any integer or step or group of integers and steps.

The term . . . at least one . . . as applied to any component of a formulation should be read to encompass one, two, three, four, or even more different occurrences of said component in the formulation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1K are pictures of exemplary formulations according to some examples of this disclosure: OPH1c (FIG. 1A), OPH4b (FIG. 1B), OPH5a (FIG. 1C), OPH1_D (FIG. 1D), OPH1_0.75D (FIGS. 1E-1F), OPH1-D/E (FIG. 1G), OPH1-D/F (FIG. 1H), OPH1-D/G (FIG. 1I), OPH1-D/H (FIG. 1J), OPH1-D/I (FIG. 1K).

FIGS. 2A-2E show droplet-size distribution (by volume) for: OPH1_0.5D (FIG. 2A), OPH1_0.75D (FIG. 2B), OPH1_D (FIG. 2C), OPH1-D/G-Placebo (FIG. 2D), and OPH1-D/G 0.3% CsA (FIG. 2E).

FIGS. 3A-3H are LUMiFuge test results for exemplary formulations loaded with cyclosporine A: OPH1_0.5D (FIG. 3A), OPH1_0.5D (FIG. 3B), OPH1_D (FIG. 3C), OPH1-D/E (FIG. 3D), OPH1-D/F (FIG. 3E), OPH1-D/G (FIG. 3F), OPH1-D/H (FIG. 3G), and OPH1-D/I (FIG. 3H).

FIG. 4 provides the Draize scoring scale for pre-clinical trials carried out on rabbits.

FIGS. 5A-5E show permeation profiles of CsA into eyes structures for OPH1c (♦), OPH5a (▪), OPH5a′ (▴) and Restasis (574 ), error bars represent SD: conjunctiva (FIG. 5A), cornea (FIG. 5B), aqueous humor (FIG. 5C), retina (FIG. 5D), and whole blood (FIG. 5E).

### DETAILED DESCRIPTION OF EMBODIMENTS

### Exemplary Formulations

Empty concentrates were prepared by weighing all concentrate components and mixing them at 40-60° C. Cyclosporin A was then solubilized into the concentrates at 50-60° C., to obtain the loaded concentrates. The aqueous phase was prepared separately, preparing first the buffer components, adjusting the pH to 7.6±0.2, followed by the addition of the polymer(s). The loaded concentrates and the aqueous phase were then combined and mixed under mild mixing conditions to obtain the final formulations, as shown in Table 1.

TABLE 1
Exemplary formulations

| | | OPH | OPH | OPH | OPH | OPH | OPH | OPH | OPH |
|---|---|---|---|---|---|---|---|---|---|
| Component | Function | 1a | 1b | 1c | 1d | 1e | 1f | 4a | 4b |
| Myrj S40* | Hydrophilic | 2.51 | 2.51 | 2.37 | 2.53 | 2.85 | 2.28 | 2.00 | 1.58 |
| Cremophor | surfactants | 2.35 | 2.48 | 2.21 | 2.36 | 2.18 | 1.75 | 1.00 | 0.79 |
| EL** | | | | | | | | | |
| HECO 40 | | 0.42 | 0.42 | 0.40 | 0.42 | 0.95 | 0.76 | 2.50 | 1.97 |
| PEG 400 | Co- | 0.59 | 0.59 | 0.55 | 0.59 | 0.67 | 0.53 | 1.20 | 0.95 |
| Propylene | surfactants | 1.26 | 1.26 | 1.18 | 1.26 | 1.42 | 1.14 | 1.80 | 1.42 |
| glycol | | | | | | | | | |
| Glycerol | Solvent | 0.59 | 0.59 | 0.55 | 0.59 | 0.67 | 0.53 | 0.50 | 0.39 |
| Castor oil | Oil | 0.25 | 0.12 | 0.24 | 0.25 | 0.28 | 0.23 | 0.50 | 0.40 |
| Cyclosporin A | API | 0.50 | 0.50 | 0.50 | 0.50 | 0.50 | 0.50 | 0.50 | 0.50 |
| PVP K30 | Film formers | 0.18 | — | 0.18 | 0.18 | 0.48 | 0.38 | — | — |
| CMC sodium | | — | 0.18 | — | — | 0.45 | 0.46 | 0.45 | 0.46 |
| Poloxamer | | — | — | — | — | 0.18 | 0.18 | 0.18 | 0.18 |
| 407 | | | | | | | | | |
| Water for | | 91.35 | 91.35 | 91.82 | 91.32 | 89.37 | 91.26 | 89.37 | 91.36 |
| injection | | | | | | | | | |

| | | | | | | | | |
|---|---|---|---|---|---|---|---|---|
| Total | 100.0 | 100.0 | 100.0 | 100.0 | 100.0 | 100.0 | 100.0 | 100.0 |
| Total concentrate | 8.47 | 8.47 | 8.00 | 8.50 | 9.52 | 7.72 | 10.00 | 8.00 |
| Total aqueous phase | 91.53 | 91.54 | 92.00 | 91.50 | 90.48 | 92.28 | 90.00 | 92.00 |

| | | OPH | OPH | OPH | OPH | OPH1 | OPH1_{—} | OPH1_{—} |
|---|---|---|---|---|---|---|---|---|
| Component | Function | 5a | 5b | 5-Ca | 5-Cb | 0.5D | 0.75D | D |
| Myrj S40* | Hydrophilic | 1.18 | 1.26 | 1.50 | 1.18 | 3.00 | 3.00 | 3.00 |
| Cremophor | surfactants | 3.16 | 3.37 | 1.50 | 1.18 | 2.96 | 2.96 | 2.96 |
| EL** | | | | | | | | |
| HECO 40*** | | — | — | 2.50 | 1.97 | 0.50 | 0.50 | 0.50 |
| PEG 400 | Co- | 0.95 | 1.01 | 1.20 | 0.95 | 0.70 | 0.70 | 0.70 |
| Propylene | surfactants | 1.42 | 1.52 | 1.80 | 1.42 | 1.50 | 1.50 | 1.50 |
| glycol | | | | | | | | |
| Glycerol | Solvent | 0.39 | 0.42 | 0.50 | 0.40 | 0.70 | 0.70 | 0.70 |
| Castor oil | Oil | 0.40 | 0.42 | 0.50 | 0.40 | 0.14 | 0.14 | 0.14 |
| CsA | API | 0.50 | 0.50 | 0.50 | 0.50 | 0.50 | 0.50 | 0.50 |
| PVP K30 | Film formers | — | — | — | — | 0.20 | 0.20 | 0.20 |
| CMC sodium | | 0.18 | 0.46 | 0.45 | 0.46 | — | — | — |
| Poloxamer | | 0.46 | 0.18 | 0.18 | 0.18 | — | — | — |
| 407 | | | | | | | | |
| NaH₂PO₄•H₂O | buffers | — | — | — | — | 0.02 | 0.03 | 0.05 |
| Na₂HPO₄ | | — | — | — | — | 0.14 | 0.20 | 0.27 |
| Water for | | 91.36 | 90.86 | 89.37 | 91.36 | 89.64 | 89.57 | 89.48 |
| injection | | | | | | | | |

| | | | | | | | |
|---|---|---|---|---|---|---|---|
| Total | 100.0 | 100.0 | 100.0 | 100.0 | 100.0 | 100.0 | 100.0 |
| Total concentrate | 8.00 | 8.50 | 10.00 | 8.00 | 10.00 | 10.00 | 10.00 |
| Total aqueous phase | 92.00 | 91.50 | 90.00 | 92.00 | 90.00 | 90.00 | 90.00 |

| | | OPH1_{—} | OPH1_{—} | OPH1_{—} | OPH1_{—} | OPH1_{—} | OPH1_{—} |
|---|---|---|---|---|---|---|---|
| Component | Function | 05D94 | D/E | D/F | D/G | D/H | D/I |
| Myrj S40* | Hydrophilic | 1.80 | 0.40 | 0.40 | 0.10 | 0.20 | 0.10 |
| Cremophor EL** | surfactants | 1.77 | 1.80 | 1.80 | 1.70 | 1.80 | 1.70 |
| HECO 40*** | | 0.30 | 0.90 | 0.90 | 0.90 | 0.90 | 0.90 |
| PEG 400 | Co- | 0.42 | 0.20 | 0.30 | 0.10 | 0.20 | 0.50 |
| Propylene glycol | surfactants | 0.90 | 1.00 | 1.00 | 1.30 | 1.30 | 1.30 |
| Glycerol | Solvent | 0.42 | 0.10 | 0.10 | 0.10 | 0.42 | 0.10 |
| Castor oil | Oil | 0.09 | 0.30 | 0.20 | 0.50 | 0.50 | 0.10 |
| CsA | API | 0.30 | 0.30 | 0.30 | 0.30 | 0.30 | 0.30 |
| PVP K30 | Film | 0.12 | 0.20 | 0.20 | 0.20 | 0.20 | 0.20 |
| | former | | | | | | |
| NaH₂PO₄•H₂O | buffers | 0.01 | 0.05 | 0.05 | 0.05 | 0.05 | 0.05 |
| Na₂HPO₄ | | 0.08 | 0.30 | 0.30 | 0.30 | 0.30 | 0.30 |
| Water for injection | | 93.79 | 94.45 | 94.45 | 94.45 | 93.86 | 94.45 |

| | | | | | | |
|---|---|---|---|---|---|---|
| Total | 100.0 | 100.0 | 100.0 | 100.0 | 100.0 | 100.0 |
| Total concentrate | 6.00 | 5.00 | 5.00 | 5.00 | 5.63 | 5.00 |
| Total aqueous phase | 94.00 | 95.00 | 95.00 | 95.00 | 94.37 | 95.00 |

*Polyoxyl 40 stearate
**Polyoxyl 35 castor oil
***Polyoxyl 40 hydrogenated castor oil

### Physical Characterization

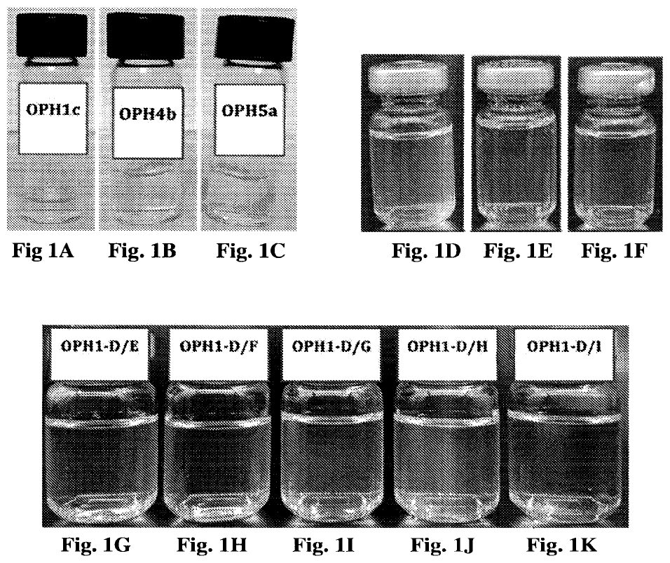

The physical properties of selected formulations are shown in Table 2. The formulations were all in the form of a dispersion of nanodomains in a continuous aqueous phase, showing high transparency, homogeneity and thermodynamic stability. OPH5a1 is non-viscosified OPH5a formulation. Exemplary visualization of the appearance of exemplary formulations can be seen in FIGS. 1A-1K.

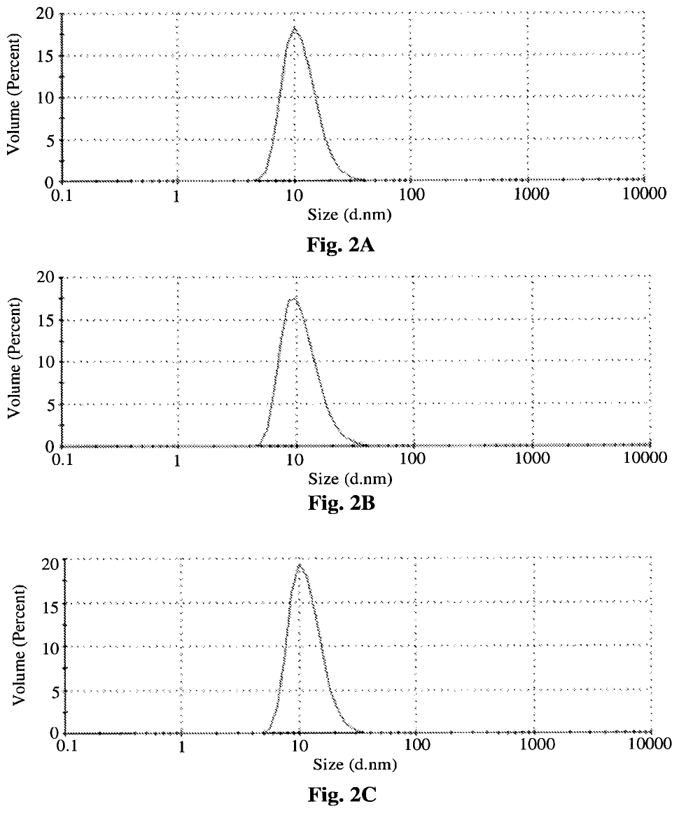

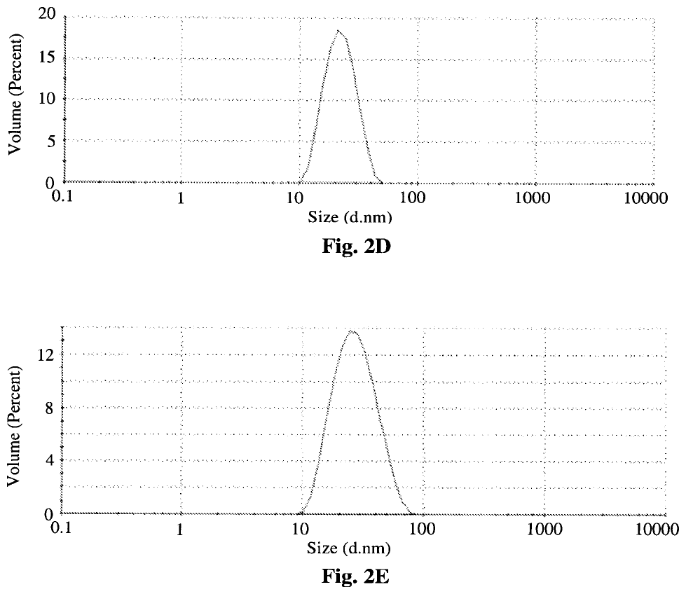

The hydrodynamic radii of the droplets were measured at room temperature by dynamic light scattering (DLS) using Nano-ZS Zetasizer (Malvern, UK), with water as a dispersant. Exemplary size distribution curves are presented in FIGS. 2A-2E.

TABLE 2
Properties of selected formulations

| Parameter | OPH1c | OPH5a | OPH5a1 | OPH1_0.5D |
|---|---|---|---|---|
| CsA content (wt %) | 0.5 | 0.5 | 0.5 | 0.5 |
| Appearance | Clear colorless | Clear colorless | Clear colorless | Clear colorless |
| | liquid | liquid | liquid | liquid |
| pH ^{a} | 6.2 | 6.4 | 6.6 | 7.5 |
| Refractive index | 1.344 | 1.345 | 1.344 | 1.346 |
| Osmolality (mOsm/Kg) ^{b} | 289 | 335 | 308 | 429 |
| Turbidity (NTU) ^{c} | 45 | 65 | 57 | — |
| Droplet size (nm) ^{d} | 10.64 (95.8%) | 14.4 (97.5%) | 14.1 (100%) | 22.5 (±1.6) |
| (volume distribution) | 244.7 (4.2%) | 388.3 (0.9%) | | |
| | | 994.1 (1.5%) | | |
| Poly Dispersion Index | 0.565 | 0.735 | 0.350 | 0.540 |
| (PDI) ^{d} | | | | |

| | | | OPH1_D/G | OPH1_D/G |
|---|---|---|---|---|
| Parameter | OPH1_0.75D | OPH1_D | Placebo | CsA-loaded |
| CsA content (wt %) | 0.5 | 0.5 | 0 | 0.3 |
| Appearance | Clear colorless | Clear colorless | Clear colorless | Clear colorless |
| | liquid | liquid | liquid | liquid |
| pH | 7.7 | 7.7 | 7.6 | 7.6 |
| Refractive index | 1.346 | 1.346 | 1.341 | 1.341 |
| Osmolality (mOsm/Kg) | — | — | 274 | 276 |
| Droplet size (nm) | 22.1 (±4.9) | 30.2 (±11.8) | 21.1 (±1.3) | 24.9 (±1.2) |
| Poly Dispersion Index (PDI) | 0.499 | 0.713 | 0.066 | 0.140 |

^{a.} pH measurements: SevenEasy Metller Toledo
^{b.} Fiske ® Micro-Osmometer (model 210)
^{c.} Turbidity evaluation: HI 83414 Turbidity and free/Total Chlorine Meter by HANNA instruments (using calibration curve samples and WFI of 0.13NTU as reference)
^{d.} Drop size examination: Zeta sizer, nano sizer (nano-s), MALVERN instrument

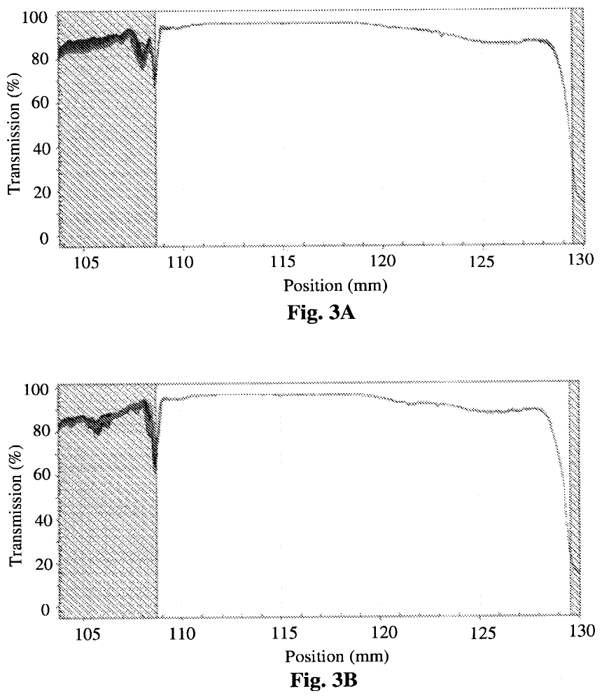

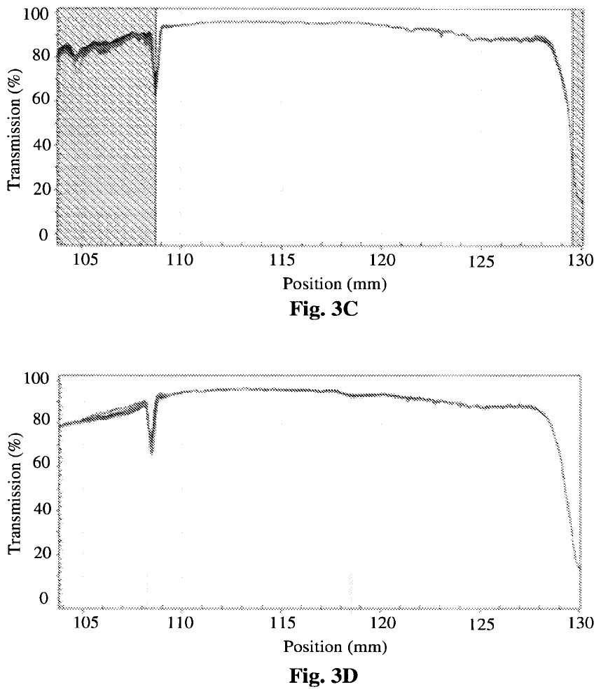

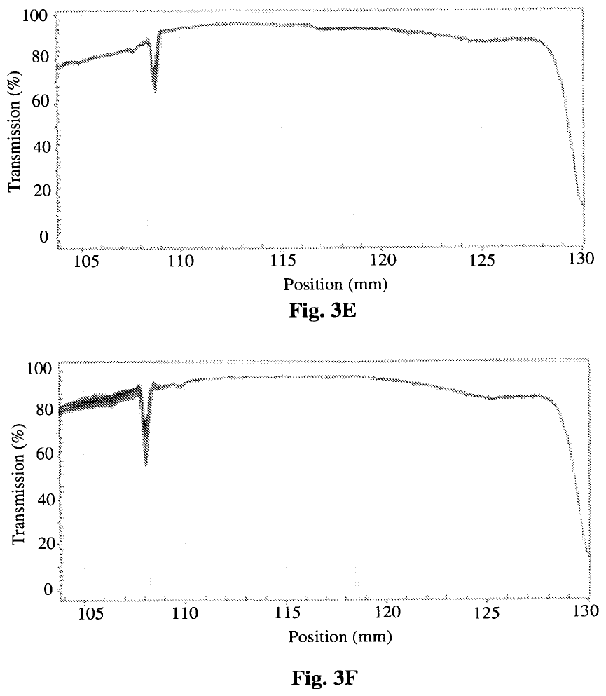

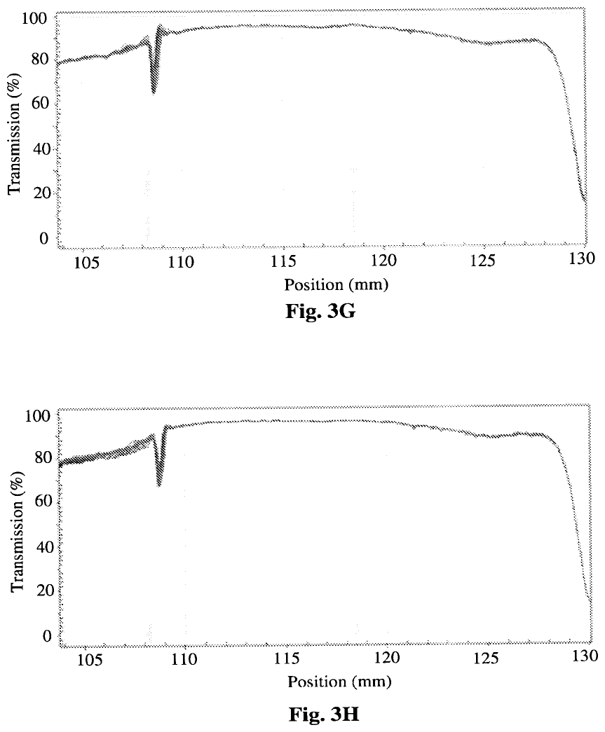

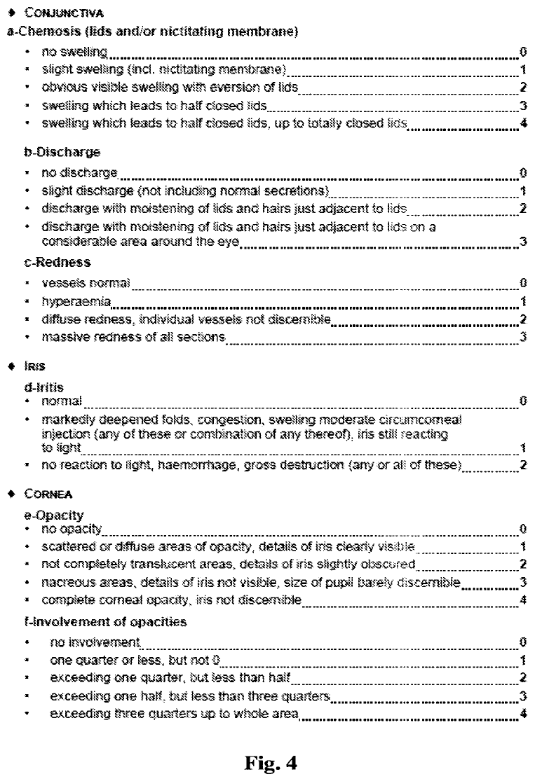

As clearly shown in Table 2, the formulations demonstrate full transparency, with an almost mono-disperse nanodroplet size and uniform refractive index. Further, as can be seen from comparing OPHI-D/G with and without cyclosporin A (CsA), the incorporation of CsA does not affect the pH, osmolality and refractive index of the formulation. The droplet size increases by about 4 nm in the presence of CsA, as can also be seen in the size-distribution curves, exhibiting wider droplets-size distribution for CsA-loaded system (FIGS. 3D-3E).

### Long-Term Physical Stability

To determine long term stability of formulations, a rapid measurement was carried out using LUMiSizer® analytical centrifugation. The results are shown in FIGS. 3A-3H. LUMiSizer® analysis enables to predict the shelf-life of a formulation in its original concentration, even in cases of slow destabilization processes like sedimentation, flocculation, coalescence and fractionation. During LUMiSizer® measurements, parallel light illuminates the entire sample cell in a centrifugal field; the transmitted light is detected by sensors arranged linearly along the total length of the sample-cell. Local alterations of particles or droplets are detected due to changes in light transmission over time. The results are presented in a graph plotting the percentage of transmitted light (Transmission %) as a function of local position (mm), revealing the corresponding transmission profile over time.

The changes in transmission indicate the stability of the formulation—when the transmission profile remains constant, the samples are considered physically stable and their shelf-life can be extrapolated based on the measurement conditions.

As shown in FIGS. 3A-3H, in all transmission profiles the lines overlap, suggesting that no changes in the transmission were observed, and all the systems are physically stable and expected, on the basis of this analysis, to be stable under storage conditions for at least 2 years.

### Chemical Stability

OPH1-0.5D was tested for chemical stability during storage at different temperatures (4° C. and 25° C.) in terms of CsA levels, visual appearance, pH, osmolality, droplet size and refractive index. The results are provided in Tables 3-1 to 3-2.

As can be seen, the formulation remains stable at the tested storage temperatures, without any evidence of change in physical and chemical properties.

TABLE 3-1
Stability for OPH1-0.5D, 4° C.

| | t₀ | | 1 month | | 3 months | |
|---|---|---|---|---|---|---|
| Appearance | Clear, transparent | | Clear, transparent | | Clear, transparent | |
| CsA Assay [HPLC] | 5.03 mg/g | | 5.06 mg/g | | 5.06 mg/g | |
| | (100.5%) | | (101.1%) | | (101.1%) | |
| Impurities | Not detected | | Not detected | | Not detected | |
| pH | 7.50 | | 7.53 | | 7.55 | |
| RI | 1.3455 | | 1.3455 | | 1.3470 | |
| Osmolality | 429 | mOsm/kg | 427 | mOsm/kg | 431 | mOsm/kg |
| Droplet Size & PDI | DS: 24.3 nm | | DS: 26.3 nm | | DS: 25.1 nm | |
| | PDI: 0.602 | | PDI: 0.488 | | PDI: 0.544 | |
| Viscosity | 2.0 | cP | 1.6 | cP | 2.0 | cP |
| | 6 months | | 9 months | | 12 months | |
| Appearance | Clear, transparent | | Clear, transparent | | Clear, transparent | |
| CsA Assay [HPLC] | 5.00 mg/g | | 5.1 mg/g | | 5.25 mg/g | |
| | (100%) | | (102.%) | | (105%) | |
| Impurities | Not detected | | Not detected | | Not detected | |
| pH | 7.53 | | 7.58 | | 7.51 | |
| RI | 1.3481 | | 1.3455 | | 1.3455 | |
| Osmolality | 425 | mOsm/kg | 430 | mOsm/kg | 428 | mOsm/kg |
| Droplet Size & PDI | DS: 30.0 nm | | DS: 26.7 nm | | DS: 26.1 nm | |
| | PDI: 0.544 | | PDI: 0.496 | | PDI: 0.477 | |
| Viscosity | 2.5 | cP | 2.5 | cP | 2.6 | cP |

TABLE 3-2
Stability for OPH1-0.5D, room temperature

| | t₀ | | 1 month | | 3 months | |
|---|---|---|---|---|---|---|
| Appearance | Clear, transparent | | Clear, transparent | | Clear, transparent | |
| CsA Assay [HPLC] | 5.03 mg/g | | 5.00 mg/g | | 5.04 mg/g | |
| | (100.5%) | | (99.9%) | | (101%) | |
| Impurities | Not detected | | Not detected | | Not detected | |
| pH | 7.50 | | 7.52 | | 7.51 | |
| RI | 1.3455 | | 1.3455 | | 1.3475 | |
| Osmolality | 429 | mOsm/kg | 428 | mOsm/kg | 428 | mOsm/kg |
| Droplet Size & PDI | DS: 24.3 nm | | DS: 28.1 nm | | DS: 29.4 nm | |
| | PDI: 0.602 | | PDI: 0.482 | | PDI: 0.536 | |
| Viscosity | 2.0 | cP | 1.5 | cP | 2.3 | cP |
| | 6 months | | 9 months | | 12 months | |
| Appearance | Clear, transparent | | Clear, transparent | | Clear, transparent | |
| CsA Assay [HPLC] | 4.95 mg/g | | 5.00 mg/g | | 5.25 mg/g | |
| | (99%) | | (100.%) | | (105%) | |
| Impurities | Not detected | | Not detected | | Not detected | |
| pH | 7.46 | | 7.46 | | 7.30 | |
| RI | 1.3461 | | 1.3455 | | 1.3455 | |
| Osmolality | 429 | mOsm/kg | 426 | mOsm/kg | 429 | mOsm/kg |
| Droplet Size & PDI | DS: 30.1 nm | | DS: 35.9 nm | | DS: 40.9 nm | |
| | PDI: 0.647 | | PDI: 0.532 | | PDI: 0.567 | |
| Viscosity | 2.2 | cP | 2.5 | cP | 2.6 | cP |

### Pharmacological Tests

All pre-clinical studies detailed below were carried out for the exemplary formulations detailed in Table 4:

TABLE 4
Selected formulations for pre-clinical studies (wt %)

| Component | OPH1c | OPH5a | OPH5a′ |
|---|---|---|---|
| Myrj S40 | 2.369 | 1.185 | 1.185 |
| Cremophor EL | 2.211 | 3.158 | 3.158 |
| HECO 40 | 0.396 | | |
| PEG 400 | 0.552 | 0.947 | 0.947 |
| Propylene glycol | 1.184 | 1.423 | 1.423 |
| Glycerol | 0.552 | 0.394 | 0.394 |
| Castor oil | 0.236 | 0.394 | 0.394 |
| CsA | 0.500 | 0.500 | 0.500 |
| PVP K30 | 0.184 | — | — |
| CMC sodium | — | 0.46 | — |
| Poloxamer 407 | — | 0.184 | — |
| Water for injection (WFI) | 91.816 | 91.356 | 92.000 |
| Total | 100.0 | 100.0 | 100.0 |

### Evaluation of Ocular Tolerability

### Study Design

The tolerability of the formulations of Table 4 was evaluated upon multiple ocular topical administration (twice a day) in male albino rabbits for five consecutive days. The treatments were instilled in conjunctival cul-de-sac of the rabbit's right eye (RE). The test design is summarized in Tables 5-1 and 5-2. At the end of the measurement period, animals treated with test items were euthanized by intracardiac injection of overdose pentobarbital following an anesthesia. Animals treated with vehicle were reused for other studies.

TABLE 5-1
Study groups and dose regimen

| | No. of | | Administration | CsA daily |
|---|---|---|---|---|
| Group | subjects | Treatment | volume per day [μL] | dosage [μg] |
| 1 | 3 | OPH1c | 50 × 2 = 100 | 250 × 2 = 500 |
| | | (0.5% CsA) | | |
| 2 | 3 | OPH5a | 50 × 2 = 100 | 250 × 2 = 500 |
| | | (0.5% CsA) | | |
| 3 | 3 | OPH5a′ | 50 × 2 = 100 | 250 × 2 = 500 |
| | | (0.5% CsA) | | |
| 4 | 3 | Vehicle | 50 × 2 = 100 | Non |
| | | (0.9% NaCl) | | |
| | | (Control) | | |

TABLE 5-2
Study schedule

| Day | Design | Ocular examination (both eyes) |
|---|---|---|
| Baseline | General clinical examination¹ | Draize examination² |
| | Body weight | |
| Day 1 to | General clinical examination¹ | Draize examination before the |
| Day 4 | Twice daily instillations RE (50 μL) | first and after the last |
| | | administration of the day |
| Day 5 | General clinical examination1 | Draize examination before the |
| | Body weight | first and after the last |
| | Twice daily instillations RE (50 μL) | administration of the day |

¹General clinical examination included the recording of general appearance and clinical signs and changes in body weight.
²An ocular examination using a light source (ophthalmoscope) followed by Draize examination of the conjunctiva, cornea and iris. See Draize scoring scale is appendix A.

### Results

The tolerability was assessed (as detailed in the design part) in terms of:
1. General behavior and body weight and
2. Ocular examination

### 1. General Behavior and Body Weight

No clinical sign of all animals was observed during the study period.

The body weight evaluation (Table 6) was normal for test items and vehicle treated animals over the five-days period. No differences in the mean body weight between the test items and vehicle treated animals was observed.

TABLE 6
Rabbits' mean body weight (±SD)

Body weight [g]

| Treatment group | Baseline | Day 5 |
|---|---|---|
| OPH1c (0.5% CsA) | 2732 (±37) | 2819 (±83) |
| OPH5a (0.5% CsA) | 2741 (±72) | 2891 (±96) |
| OPH5a′ (0.5% CsA) | 2741 (±56) | 2907 (±92) |
| Vehicle (0.9% NaCl) | 2788 (±112) | 2806 (±247) |

### 2. Ocular Examination

Individual data of ocular examination on both eyes are summarized in Table 7. Effects in Table 7 are provided according to the Draize scoring scale (FIG. 4).

TABLE 7
Ocular findings for all treatment groups

| | | Treatment (n = 3 treated eyes/group) | | | |
|---|---|---|---|---|---|
| | | OPH1c | OPH5a | OPH5a′ | Vehicle |
| Eye structure | | (0.5% CsA) | (0.5% CsA) | (0.5% CsA) | (0.9% NaCl) |
| Conjunctiva | Indication¹ | — | — | Redness | — |
| | Score² | — | — | 1/3 | — |
| | Comments | — | — | One RE on Day | — |
| | | | | 5 before the first | |
| | | | | administration | |
| Iris | Indication¹ | Iritis | Iritis | Iritis | — |
| | Score² | 1/2 | 1/2 | 1/2 | |
| | Comments | One RE on Day 2 | One RE on Day | One RE on Day | — |
| | | after last | 1 after last | 2 after last | |
| | | administration | administration | administration | |
| Cornea | Indication¹ | — | — | — | — |
| | Score² | — | — | — | — |
| | Comments | — | — | — | — |

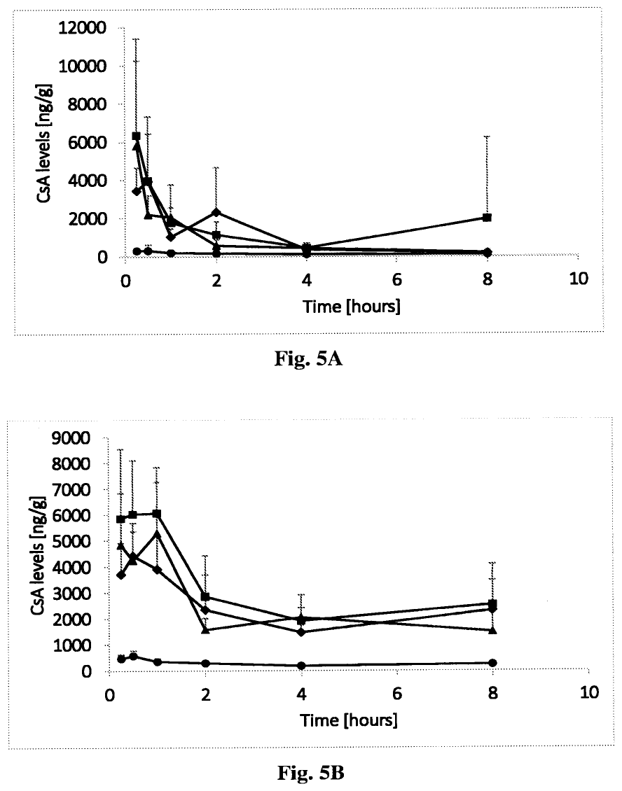

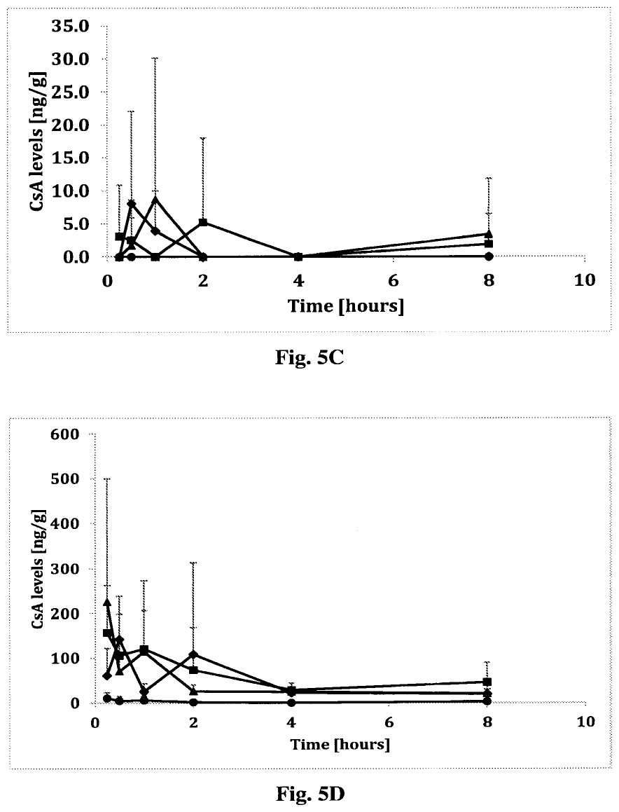

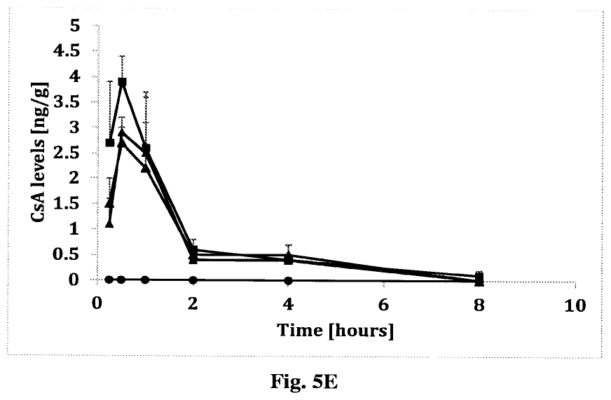

¹Indications are listed in FIG. 5
²The score was given according to the indication as detailed in Draize scale and is denoted n/nₘₐₓ. For instance, the indication of iritis can get a score of 1/2 or 2/2.

As seen in Table 7, ocular findings, where observed, occurred only in the treated eye (right eye). Vehicle treatment group showed no ocular findings, while for the other treatment groups only slight and transient ocular findings were recorded, indicating that the three tested formulations are macroscopically tolerated as the vehicle.

Overall, all three tested formulations were found to be tolerable under the experimental conditions of multiple topical administration over consecutive 5 days.

### Evaluation of Ocular Penetration of CsA

### Study Objective and Design

The aim of this study was to evaluate the permeation of CsA into conjunctiva (CJ), cornea (C), aqueous humor (AH), retina (R) and whole blood (WB) after a single conjunctival cul-de-sac instillation of 50 μL of the tested formulations in both eyes of pigmented rabbits (HY79b strain), in comparison to commercially available product of Restasis (contains 0.05% CsA).

For the purpose of the study 72 pigmented rabbits were divided into four group of 18 subjects, which were further sub-divided into 6 time points (Table 8).

TABLE 8
Study groups and dose regimen

| | No. | No. | | | |
|---|---|---|---|---|---|
| | Rabbit | Rabbit | | CsA | Time- |
| Treatment | per | per | Administration | dosage | points |
| group | group | timepoint | volume [μL] | [μg] | [hr] |
| OPH1c | 18 | 3 | 50 | 500 | 0.25, 0.5, |
| (0.5% CsA) | | | | | 1, 2, 4, 8 |
| OPH5a | 18 | 3 | 50 | 500 | 0.25, 0.5, |
| (0.5% CsA) | | | | | 1, 2, 4, 8 |
| OPH5a′ | 18 | 3 | 50 | 500 | 0.25, 0.5, |
| (0.5% CsA) | | | | | 1, 2, 4, 8 |
| Restasis | 18 | 3 | 50 | 50 | 0.25, 0.5, |
| (0.05% CsA) | | | | | 1, 2, 4, 8 |

Both treated eyes of each rabbit were examined using a light source (Draize's scale, FIG. 4) at baseline and at the end point. At all time points, animals were anesthetized by an intramuscular injection of mixed solution of Rompun® (xylazine) and Imalgene® 1000 (ketamine). Whole blood was sampled into K3-EDTA anticoagulant tubes by intracardiac puncture before animal euthanasia and stored at −80±15° C. Then animals were euthanaized by intracardiac injection of overdosed pentobarbital. Immediately after euthanasia, cornea, conjunctiva, aqueous humor and retina were dissected from both eyes, weighed and stored at −80±15° C. CsA was extracted from different structures of both eyes and whole blood and its content was determined by RRLC-MS/MS. The Analyzed CsA levels were further used to calculate the PK parameters including apparent Cmax, apparent Tmax and AUC_{0.25-8}hr. PK parameters were calculated using the mean values of the group.

### Results

The penetration profile of CsA into CJ, C, AH, R and WB are presented in Tables 9-1 to 9-5 (as well as FIGS. 5A-5E), PK results are shown in Table 10.

TABLE 9-1
CsA permeation into conjunctiva for all treatment groups

| | CsA concentration in conjunctiva (ng/g) | | | | | | | | | | | |
|---|---|---|---|---|---|---|---|---|---|---|---|---|
| Time | OPH1c | | | OPH5a | | | OPH5a′ | | | Restasis | | |
| (hr) | Mean | SD | N | Mean | SD | N | Mean | SD | N | Mean | SD | N |
| 0.25 | 3440 | 1215 | 6 | 6347 | 3920 | 6 | 5800 | 5598 | 6 | 295 | 93 | 6 |
| 0.5 | 3945 | 3390 | 6 | 3975 | 2466 | 6 | 2196 | 997 | 6 | 304 | 301 | 6 |
| 1 | 1044 | 432 | 6 | 1790 | 773 | 6 | 2057 | 1715 | 6 | 196 | 111 | 6 |
| 2 | 2321 | 2354 | 6 | 1151 | 687 | 6 | 562 | 329 | 6 | 156 | 68 | 6 |
| 4 | 358 | 240 | 6 | 449 | 138 | 6 | 431 | 277 | 6 | 116 | 93 | 6 |
| 8 | 167 | 156 | 6 | 1983 | 4243 | 6 | 197 | 79 | 6 | 8 | 77 | 6 |

TABLE 9-2
CsA permeation into cornea for all treatment groups

| | CsA concentration in cornea (ng/g) | | | | | | | | | | | |
|---|---|---|---|---|---|---|---|---|---|---|---|---|
| Time | OPH1c | | | OPH5a | | | OPH5a′ | | | Restasis | | |
| (hr) | Mean | SD | N | Mean | SD | N | Mean | SD | N | Mean | SD | N |
| 0.25 | 3725 | 1183 | 6 | 5858 | 983 | 6 | 4836 | 3719 | 6 | 465 | 165 | 6 |
| 0.5 | 4422 | 929 | 6 | 6030 | 2080 | 6 | 4242 | 1441 | 6 | 575 | 205 | 6 |
| 1 | 3919 | 1301 | 6 | 6067 | 1203 | 6 | 5291 | 2558 | 6 | 354 | 123 | 6 |
| 2 | 2350 | 1351 | 6 | 2861 | 1569 | 6 | 1573 | 452 | 6 | 289 | 88 | 6 |
| 4 | 1472 | 287 | 6 | 1925 | 497 | 6 | 2056 | 866 | 6 | 181 | 42 | 6 |
| 8 | 2325 | 1163 | 6 | 2542 | 1557 | 6 | 1503 | 752 | 6 | 242 | 74 | 6 |

TABLE 9-3
CsA permeation into aqueous humor for all treatment groups

| | CsA concentration in aqueous humor (ng/g) | | | | | | | | | | | |
|---|---|---|---|---|---|---|---|---|---|---|---|---|
| Time | OPH1c | | | OPH5a | | | OPH5a′ | | | Restasis | | |
| (hr) | Mean | SD | N | Mean | SD | N | Mean | SD | N | Mean | SD | N |
| 0.25 | 0.0 | 0.0 | 6 | 3.1 | 7.7 | 6 | 0.0 | 0.0 | 6 | 0.0 | 0.0 | 6 |
| 0.5 | 8.0 | 14.1 | 6 | 2.5 | 6.1 | 6 | 1.7 | 4.2 | 6 | 0.0 | 0.0 | 6 |
| 1 | 3.9 | 6.1 | 6 | 0.0 | 0.0 | 6 | 8.7 | 21.4 | 6 | 0.0 | 0.0 | 6 |
| 2 | 0.0 | 0.0 | 6 | 5.2 | 12.8 | 6 | 0.0 | 0.0 | 6 | 0.0 | 0.0 | 6 |
| 4 | 0.0 | 0.0 | 6 | 0.0 | 0.0 | 6 | 0.0 | 0.0 | 6 | 0.0 | 0.0 | 6 |
| 8 | 0.0 | 0.0 | 6 | 1.9 | 4.6 | 6 | 3.4 | 8.4 | 6 | 0.0 | 0.0 | 4 |

TABLE 9-4
CsA permeation into retina for all treatment groups

| | CsA concentration in retina (ng/g) | | | | | | | | | | | |
|---|---|---|---|---|---|---|---|---|---|---|---|---|
| Time | OPH1c | | | OPH5a | | | OPH5a′ | | | Restasis | | |
| (hr) | Mean | SD | N | Mean | SD | N | Mean | SD | N | Mean | SD | N |
| 0.25 | 60.8 | 60.9 | 6 | 157.6 | 104.9 | 6 | 225.3 | 273.8 | 6 | 10.4 | 12.6 | 6 |
| 0.5 | 142.5 | 55.8 | 6 | 105.8 | 132.4 | 6 | 69.9 | 31.8 | 6 | 4.1 | 10 | 6 |
| 1 | 24.6 | 17.9 | 6 | 120.5 | 152.4 | 114.1 | 92.6 | 21.4 | 6 | 5.8 | 9.1 | 6 |
| 2 | 108.3 | 205.1 | 6 | 73.6 | 95.2 | 6 | 25.4 | 13.9 | 6 | 1.6 | 3.8 | 6 |
| 4 | 22.0 | 5.2 | 6 | 28.1 | 15.6 | 6 | 24.3 | 19.9 | 6 | 0.0 | 0.0 | 6 |
| 8 | 19.1 | 10.3 | 6 | 44.9 | 45.5 | 6 | 20.0 | 20.4 | 6 | 2.5 | 6.2 | 6 |

TABLE 9-5
CsA permeation into whole blood for all treatment groups

| | CsA concentration in whole blood (ng/g) | | | | | | | | | | | |
|---|---|---|---|---|---|---|---|---|---|---|---|---|
| Time | OPH1c | | | OPH5a | | | OPH5a′ | | | Restasis | | |
| (hr) | Mean | SD | N | Mean | SD | N | Mean | SD | N | Mean | SD | N |
| 0.25 | 1.5 | 0.5 | 3 | 2.7 | 1.2 | 3 | 1.1 | 0.5 | 3 | 0.0 | 0.0 | 3 |
| 0.5 | 2.7 | 0.3 | 3 | 3.9 | 0.5 | 3 | 2.9 | 0.3 | 2 | 0.0 | 0.0 | 3 |
| 1 | 2.2 | 0.9 | 3 | 2.6 | 1.1 | 3 | 2.5 | 1.1 | 3 | 0.0 | 0.0 | 2 |
| 2 | 0.5 | 0.1 | 3 | 0.6 | 0.2 | 3 | 0.4 | 0.0 | 3 | 0.0 | 0.0 | 3 |
| 4 | 0.5 | 0.2 | 2 | 0.4 | 0.1 | 3 | 0.4 | 0.1 | 3 | 0.0 | 0.0 | 3 |
| 8 | 0.0 | 0.0 | 3 | 0.1 | 0.1 | 3 | 0.0 | 0.0 | 3 | 0.0 | 0.0 | 3 |

TABLE 10
PK parameters obtained for all tested groups

| Eye structure | PK parameter | OPH1c | OPH5a | OPH5a′ | Restasis |
|---|---|---|---|---|---|
| Conjunctiva | Cmax [ng/g] | 3945 | 6347 | 5800 | 304 |
| | Tmax [hours] | 0.5 | 0.25 | 0.25 | 0.5 |
| | AUC_{0.25-8} [ng/g · hr] | 7582 | 10664 | 5621 | 1078 |
| | % AUC_{0.25-8} from | 3.03 | 4.27 | 2.25 | 4.31 |
| | applied dose | | | | |
| Cornea | Cmax [ng/g] | 4422 | 6067 | 5291 | 575 |
| | Tmax [hours] | 0.5 | 1 | 1 | 0.5 |
| | AUC_{0.25-8} [ng/g · hr] | 17654 | 22693 | 17696 | 1999 |
| | % AUC_{0.25-8} from | 7.06 | 9.08 | 7.08 | 8.00 |
| | applied dose | | | | |
| Aqueous | Cmax [ng/g] | 8.0 | 5.2 | 8.7 | 0.0 |
| humor | Tmax [hours] | 0.5 | 2.0 | 1.0 | NA |
| | AUC_{0.25-8} [ng/g · hr] | 6.0 | 12.9 | 14.1 | 0.0 |
| | % AUC_{0.25-8} from | 0.002 | 0.005 | 0.006 | 0.000 |
| | applied dose | | | | |
| Retina | Cmax [ng/g] | 142.5 | 157.6 | 225.3 | 10.4 |
| | Tmax [hours] | 0.5 | 0.25 | 0.25 | 0.25 |
| | AUC_{0.25-8} [ng/g · hr] | 346.2 | 434.3 | 291.0 | 14.5 |
| | % AUC_{0.25-8} from | 0.14 | 0.17 | 0.12 | 0.06 |
| | applied dose | | | | |
| Whole Blood | Cmax [ng/g] | 2.7 | 3.9 | 2.9 | 0.0 |
| | Tmax [hours] | 0.5 | 0.5 | 0.5 | NA |
| | AUC_{0.25-8} [ng/g · hr] | 5.1 | 5.8 | 4.8 | 0.0 |
| | % AUC_{0.25-8} from | 0.002 | 0.002 | 0.002 | 0.000 |
| | applied dose | | | | |

Based on the Draize scaling, all tested formulations as well as Restasis are microscopically tolerable.

According to the permeation profiles and the calculated PK parameters, mostly AUC values that provide information on the accumulated permeation of CsA upon the period of exposure, we can summarize: The penetration of CsA into the conjunctiva in absolute terms was found to be in the following order: OPH5a>OPHIc>OPH5a′>Restasis.

However, in terms of % from applied dose Restasis showed the highest permeation following by OPH5a, OPHIc and final OPH5a′.

The penetration of CsA into the cornea in absolute terms was found to be in the following order: OPH5a>OPH5a′≈OPH1c>Restasis (or in terms of % from applied dose: OPH5a>Restasis>OPH5a′≈OPHIc).

The penetration of CsA into the aqueous humor in absolute terms was found to be in the following order: OPH5a>OPH5a′>OPH1c>Restasis.

The penetration of CsA into the retina and to the whole blood in absolute terms was found to be in the following order: OPH5a>OPH1c>OPH5a′>Restasis.

Overall, there is high bioavailability of all three tested formulations to the retina compared to Restasis.

1.-42. (canceled)

43. An ophthalmic formulation comprising plurality of nanostructures dispersed in an aqueous continuous phase, the nanostructures being in the form of droplets having an average diameter of at most 50 nm, the nanostructures comprising:
a) cyclosporin in a concentration of at least 0.1 wt % of the formulation,
b) at least two non-ionic hydrophilic surfactants,
c) at least one oil in a concentration of at most 2 wt % of the formulation, and
d) at least one co-surfactant.

44. The ophthalmic formulation of claim 43, wherein the said at least two non-ionic hydrophilic surfactants comprise at least one first non-ionic hydrophilic surfactant selected from the group consisting of ethoxylated fatty acids, and at least one second non-ionic hydrophilic surfactant selected from the group consisting of ethoxylated castor oil and hydrogenated derivatives thereof.

45. The ophthalmic formulation of claim 44, wherein the weight ratio of the first non-ionic hydrophilic surfactants to the second non-ionic hydrophilic surfactants ranges between about 1:1 and 1:30.

46. The ophthalmic formulation of claim 43, wherein the total concentration of the non-ionic hydrophilic surfactants in the formulation ranges between about 1 wt % and about 7 wt %.

47. The ophthalmic formulation of claim 43, wherein said at least one oil is selected from the group consisting of acylglycerides of fatty acids, triacetin, tributyrin, tricaprylin, triolein, medium chain triglyceride and mixed fatty acids triglycerides, olive oil, sesame oil, soybean oil, canola oil, castor oil, partially or fully hydrogenated castor oil, paraffin oil, mineral oil, non-saponified fatty derivatives, alkyl alcohols including oleyl alcohol, dodecyl alcohol, terpenoids, and combinations thereof.

48. The ophthalmic formulation of claim 43, wherein said at least one oil is present in the formulation in a concentration of no more than 0.7 wt %.

49. The ophthalmic formulation of claim 43, wherein said at least one co-surfactant is present in the formulation in a concentration ranging between about 0.5 wt % and 5 wt %, and the weight ratio between the non-ionic hydrophilic surfactants and the co-surfactants ranges between about 1:1 and about 5:1.

50. The ophthalmic formulation of claim 43, wherein the nanostructures further comprise at least one solvent.

51. The ophthalmic formulation of claim 50, wherein the formulation comprises said at least one solvent in a concentration ranging between about 0.05 wt % and about 1.5 wt %, and the weight ratio between the non-ionic hydrophilic surfactants and the solvents ranges between about 5:1 and about 35:1.

52. The ophthalmic formulation of claim 43, wherein said aqueous phase comprises at least one film forming agent.

53. The ophthalmic formulation of claim 52, wherein the concentration of said at least one film forming agent in the formulation is up to about 0.75 wt %.

54. The ophthalmic formulation of claim 43, wherein the droplets are substantially mono-disperse.

55. A cyclosporin concentrate formulation for preparing the ophthalmic formulation of claim 43, the concentrate comprising:
a) cyclosporin in a concentration of at least 0.5 wt % of the concentrate,
b) at least two non-ionic hydrophilic surfactants,
c) at least one oil in a concentration of at most 12 wt % of the concentrate, and
d) at least one co-surfactant.

56. The concentrate formulation of claim 55, wherein the total concentration of the non-ionic hydrophilic surfactants in the concentrate ranges between about 20 wt % and about 75 wt %.

57. The concentrate formulation of claim 55, wherein said at least one oil is present in the concentrate in a concentration of no more than 7 wt %.

58. The concentrate formulation of claim 55, wherein said at least one co-surfactant is present in the concentrate in a concentration ranging between about 20 wt % and about 45 wt %, and wherein the weight ratio between the non-ionic hydrophilic surfactants and the co-surfactants ranges between about 1:1 and about 5:1.

59. The concentrate formulation of claim 55, wherein the nanostructures further comprise at least one solvent in a concentration ranging between about 1 wt % and about 10 wt %, and wherein the weight ratio between the non-ionic hydrophilic surfactants and the solvents ranges between about 5:1 and about 35:1.

60. The concentrate formulation of claim 55, being substantially devoid of water.

61. A kit for preparing the ophthalmic formulation of claim 43, comprising:
at least one first container containing the concentrate of claim 55;
at least one second container containing an aqueous dispersing medium; and
instructions for use.

62. The kit of claim 61, wherein said first container and said second container are integrally formed and configured for mixing said concentrate and aqueous dispersing medium upon user demand.
